(19) United States
(12) Patent Application Publication
(10) Pub. No.: US 2025/0107925 A1
(43) Pub. Date: Apr. 3, 2025

(54) PLANT-BASED SUBSTRATE, APPLICATIONS AND METHOD OF FABRICATING THEREOF

(71) Applicants: Nanyang Technological University, Singapore (SG); Purdue Research Foundation, West Lafayette, IN (US)

(72) Inventors: Nam-Joon CHO, Singapore (SG); Subra SURESH, Singapore (SG); Chi Hwan LEE, West Lafayette, IN (US); Youngkyu HWANG, Singapore (SG); Min Ku KIM, West Lafayette, IN (US)

(21) Appl. No.: 18/725,571

(22) PCT Filed: Jan. 5, 2023
(86) PCT No.: PCT/SG2023/050008

§ 371 Date: Jun. 28, 2024

Related U.S. Application Data
(60) Provisional application No. 63/297,386, filed on Jan. 7, 2022.

(51) Int. Cl. A61F 7/08; A61F 7/00; A61F 7/02; H01B 1/02; H05B 3/34

(52) U.S. Cl. CPC A61F 7/08; H01B 1/02; H05B 3/34; A61F 2007/0035; A61F 2007/0078; A61F 2007/0207; H05B 2203/013; H05B 2203/017

(57) ABSTRACT

Disclosed herein is a conductive composite material, comprising a pollen-based substrate layer, an elastomeric adhesive layer on top of the pollen-based substrate layer, a biocompatible polymer substrate layer on top of the elastomeric adhesive layer, and a metal layer on top of the biocompatible polymer substrate layer and a method of forming the same. Also disclosed herein is a stretchable biopolymer-based heating pad comprising a pollen-based substrate layer, a cured polymeric layer or a metal layer on top of the pollen-based substrate layer, where, when present, the cured polymeric layer comprises a cured polymeric material and a metal, an encapsulation coating comprising a polymeric material that encapsulates the cured polymeric layer or the metal layer, wherein the pollen-based substrate layer and the cured polymeric layer are patterned to provide a heating pad and the method of forming the same.

### FIELD OF INVENTION

This invention relates to a biopolymer/plant-based substrate for flexible and stretchable electronics and a method of fabricating the same. The biopolymer/plant-based substrate may be from plant pollen.

### BACKGROUND

The listing or discussion of a prior-published document in this specification should not necessarily be taken as an acknowledgement that the document is part of the state of the art or is common general knowledge.

The development of ultrathin, portable, and flexible electronic devices and components has catalyzed rapid technological advances across a wide range of domains, including medicine, telecommunication, energy, and robotics. Synthetic polymers such as polyimide (PI), polystyrene, polypropylene, and polyethylene terephthalate (PET) are widely used as substrate materials in applications that incorporate transistors, light-emitting diodes, biosensors, and organic photovoltaics. However, the use of these chemically processed synthetic polymers, which are non-reusable and non-degradable, can lead to environmental degradation on land and in oceans. Consequently, the development and deployment of materials derived from abundant natural resources that are sustainable, eco-friendly, biocompatible, and biodegradable are receiving increasing attention.

Researchers have explored materials such as chitin, nanocellulose, and silk as substrate materials to replace synthetic chemicals in electronics applications. For example, chitin, a fibrous material, has been considered a candidate substrate material for organic light-emitting diodes (OLEDs), glucose sensors, and heaters owing to its robustness, non-toxicity, and biocompatibility. In addition, nanocellulose-based substrates with a low surface roughness, high transparency, and high elastic modulus (E=6-17 GPa) have been developed for use in energy storage devices, OLEDs, and organic photovoltaics. Notably, the extraction of chitin and cellulose from diverse sources such as crabs, wood, algae, and bacteria is challenging in terms of the high extraction costs, low yield, and considerable processing time. Silk-based substrates exhibit high mechanical strength (E=5-12 GPa) and can be conveniently fabricated in large volumes. However, despite its excellent biocompatibility and biodegradability, silk is highly soluble in water, which limits its long-term reliable use in flexible and stretchable electronics.

Thus, there exists a need for alternatives to synthetic materials for electronic applications and more specifically for flexible and stretchable electronics.

### SUMMARY OF INVENTION

It has been surprisingly found that a material that solves some or all of the problems discussed hereinbefore can be manufactured based on a pollen-based substrate material.

Aspects and embodiments of the invention will now be discussed by reference to the following non-limiting clauses.

1. A conductive composite material, comprising:
a pollen-based substrate layer;
an elastomeric adhesive layer on top of the pollen-based substrate layer;
a biocompatible polymer substrate layer on top of the elastomeric adhesive layer; and a metal layer on top of the biocompatible polymer substrate layer.

2. The conductive composite material according to Clause 1, wherein the pollen-based substrate comprises a plurality of pollen microgels.

3. The conductive composite material according to Clause 2, wherein the plurality of pollen microgels are derived from pollen grains from one or more of the group selected from sunflower (Helianthus annuus L.) pollen grains, pine (Pinus taeda) pollen grains, daisy (Baccharis halimifolia L.) pollen grains, cattail (Typhae angustfolia) pollen grains, camellia (Camellia Sinensis L.) pollen grains, bee pollen grains, and lycopodium (Lycopodium clavatum) spores (S-type).

4. The conductive composite material according to any one of the preceding clauses, wherein the elastomeric adhesive layer on top of the pollen-based substrate layer is formed from a silicone elastomer or an acrylic adhesive.

5. The conductive composite material according to Clause 4, wherein the elastomeric adhesive layer on top of the pollen-based substrate layer is a platinum-cured silicone elastomer.

6. The conductive composite material according to any one of the preceding clauses, wherein the biocompatible polymer substrate layer on top of the elastomeric adhesive layer is formed from one or more of polyimide, polyethylene terephthalate (PET), a polyurethane, and a parylene.

7. The conductive composite material according to Clause 6, wherein the biocompatible polymer substrate layer on top of the elastomeric adhesive layer is formed from polyimide.

8. The conductive composite material according to any one of the preceding clauses, wherein the metal layer on top of the biocompatible polymer substrate layer is formed from one or more of the group consisting of chromium (Cr), titanium (Ti), copper (Cu), platinum (Pt), silver (Ag), and gold (Au).

9. The conductive composite material according to Clause 8, wherein the metal layer on top of the biocompatible polymer substrate layer is presented as a first layer and a second layer, with the first layer in direct contact with the biocompatible polymer substrate layer.

10. The conductive composite material according to Clause 9, wherein the first layer is formed from one or more of chromium (Cr), titanium (Ti), and copper (Cu), and the second layer is formed from one or more of platinum (Pt), silver (Ag), and gold (Au), optionally wherein the first layer is chromium (Cr) and the second layer is gold (Au).

11. The conductive composite material according to Clause 9 or Clause 10, wherein the first layer has a thickness of from 1 to 50 nm, such as from 2 to 25 nm, such as about 5 nm and the second layer has a thickness of from 100 to 250 nm, such as from 125 to 200 nm, such as about 150 nm.

12. The conductive composite material according to any one of the preceding clauses, wherein the metal layer is patterned to provide electrodes.

13. The conductive composite material according to any one of the preceding clauses, wherein the adhesive layer is an acrylic adhesive.

14. A method of forming a conductive composite material as described in any one of Clauses 1 to 13, the method comprising the steps of:
(ai) providing a pollen-based substrate layer and a conductive intermediate comprising:
   a biocompatible polymer substrate layer; and
   a metal layer on top of the biocompatible polymer substrate layer; and
(aii) attaching the conductive intermediate to the pollen-based substrate layer by an elastomeric adhesive to provide the conductive composite material,
   optionally wherein the metal layer is patterned to provide an electrode.

15. The method according to Clause 14, wherein the conductive intermediate is provided by the steps of:
(bi) depositing a layer of polymethyl methacrylate (PMMA) on a substrate to provide a PMMA-coated substrate;
(bii) depositing a layer of a biocompatible polymer on the PMMA-coated substrate;
(biii) depositing a metal layer on the biocompatible polymer layer; and
(biv) immersing the PMMA layer in acetone to dissolve the PMMA layer and release the conductive intermediate.

16. The method according to Clause 15, wherein the metal layer if formed from a first metal layer and a second metal layer, where the first metal layer is formed from one or more of chromium (Cr), Titanium (Ti), and Copper (Cu), and the second metal layer is formed from one or more of Platinum (Pt), Silver (Ag), and gold (Au), optionally wherein the first layer is chromium (Cr) and the second layer is gold (Au).

17. The method according to Clause 16, wherein the first metal layer has a thickness of from 1 to 50 nm, such as from 2 to 25 nm, such as about 5 nm and the second metal layer has a thickness of from 100 to 250 nm, such as from 125 to 200 nm, such as about 150 nm.

18. A stretchable biopolymer-based heating pad, comprising:
a pollen-based substrate layer;
a cured polymeric layer or a metal layer on top of the pollen-based substrate layer, where, when present, the cured polymeric layer comprises a cured polymeric material and a metal; and
an encapsulation coating comprising a polymeric material that encapsulates the cured polymeric layer or the metal layer, wherein
the pollen-based substrate layer and the cured polymeric layer are patterned to provide a heating pad.

19. The stretchable biopolymer-based heating pad according to Clause 18, wherein the encapsulation coating further encapsulates the pollen-based substrate layer.

20. The stretchable biopolymer-based heating pad according to Clause 18 or Clause 19, wherein, when present, the metal layer on top of the pollen-based substrate layer is formed from one or more of the group consisting of chromium (Cr), titanium (Ti), copper (Cu), platinum (Pt), silver (Ag), and gold (Au).

21. The stretchable biopolymer-based heating pad according to Clause 20, wherein the metal layer on top of the pollen-based substrate layer is presented as a first layer and a second layer, with the first layer in direct contact with the pollen-based substrate layer, optionally wherein:
(aa) the first layer is formed from one or more of chromium (Cr), titanium (Ti), and copper (Cu), and the second layer is formed from one or more of platinum (Pt), silver (Ag), and gold (Au), optionally wherein the first layer is chromium (Cr) and the second layer is gold (Au); and/or
(ab) the first layer has a thickness of from 1 to 50 nm, such as from 2 to 25 nm, such as about 5 nm and the second layer has a thickness of from 100 to 250 nm, such as from 125 to 200 nm, such as about 150 nm.

22. The stretchable biopolymer-based heating pad according to any one of Clauses 18 to 21, wherein the pollen-based substrate comprises a plurality of pollen microgels.

23. The stretchable biopolymer-based heating pad according to Clause 22, wherein the plurality of pollen microgels are derived from pollen grains from one or more of the group selected from sunflower (Helianthus annuus L.) pollen grains, pine (Pinus taeda) pollen grains, daisy (Baccharis halimifolia L.) pollen grains, cattail (Typhae angustfolia) pollen grains, camellia (Camellia Sinensis L.) pollen grains, bee pollen grains, and lycopodium (Lycopodium clavatum) spores (S-type).

24 The stretchable biopolymer-based heating pad according to any one of Clauses 18, 19 and 22 to 23, wherein, when present, the cured polymeric material is selected from one or more of a polyurethane and a silicone elastomer, optionally wherein the cured polymeric material is a polyurethane.

25. The stretchable biopolymer-based heating pad according to any one of Clauses 18, 19 and 22 to 24, wherein, when the cured polymeric material is present, the metal in the cured polymeric material is selected from one or more of the group consisting of platinum (Pt), silver (Ag), and gold (Au), optionally wherein the metal is silver (Ag).

26. The stretchable biopolymer-based heating pad according to any one of Clauses 18 to 25, wherein the encapsulation coating is a silicone elastomer, optionally wherein the encapsulation coating is polydimethylsiloxane (PDMS).

27. A method of forming a stretchable biopolymer-based heating pad as described in any one of Clauses 18 to 26, wherein the method comprises the steps of:
(ci) providing a heating pad intermediate comprising:
   a pollen-based substrate layer;
   a cured polymeric layer or a metal layer on top of the pollen-based substrate layer, where, when present, the cured polymeric layer comprises a cured polymeric material and a metal; and
(cii) encapsulating at least the cured polymeric layer or the metal layer on top of the pollen-based substrate layer with an encapsulation coating comprising a polymeric material, optionally wherein the encapsulation coating is a silicone, further optionally wherein the encapsulation coating is polydimethylsiloxane (PDMS).

28. The method according to Clause 27, wherein the encapsulating step (cii) further encapsulates the pollen-based substrate layer.

29. The method according to Clause 27 or Clause 28, wherein the heating pad intermediate is formed by the steps of:
(di) providing a patterned pollen-based substrate layer that has been patterned to provide the shape of a heating pad; and
(dii) applying a polymeric layer comprising a polymeric material and a metal onto the patterned pollen-based substrate layer and curing the polymeric layer after it has been applied or applying a metal layer onto the patterned pollen-based substrate layer.

30. The method according to Clause 29, wherein the patterned pollen-based substrate layer is formed by:
(ei) providing a pollen-based substrate that has not been patterned; and
(eii) patterning the pollen-based substrate to provide the shape of a heating pad.

### BRIEF DESCRIPTION OF FIGURES

FIG. 1 is a schematic illustration of the process to fabricate the pollen-based substrate.

FIG. 2 includes (a) scanning electron microscope (SEM) images and (b) a graphical illustration of the Young's modulus of a sample of sunflower (Helianthus annuus L.) pollen grains, camellia (Camellia Sinensis L.) pollen grains and lycopodium (Lycopodium clavatum) spores.

FIG. 3 is a FT-IR spectrum of the camellia pollen grains during the substrate fabrication process (i.e., untreated, after being defatted and after being treated with KOH).

FIGS. 4(a), (b) and (c) are SEM images depicting the morphology of the bee pollen grains from the camellia during the substrate fabrication process (i.e., (a) untreated (b) after being defatted and (c) after being treated with KOH). FIG. 4(d) is a photograph of the resulting pollen-based substrate obtained from the fabrication process with portions on the top and bottom sides identified for taking SEM images. FIGS. 4(e) and 4(f) are SEM images (including magnified portions) of the top and bottom sides identified on the pollen-based substrate of FIG. 4(d) respectively.

FIG. 5(a) is a strain-stress curve of the pollen-based substrate compared to a commercial A4 paper (PaperOne™, April Group). FIG. 5(b) is a photograph of a crane origami folded from the pollen-based substrate. FIGS. 5(c), (d), (e) and (f) depict the higher chemical resistance of the pollen-based substrate, as compared to polycarbonate (PC), polyethylene terephthalate (PET), and polyimide (PI) films, to various solvents (i.e., deionized (DI) water, 2 M NaOH, 2 M HCl, and acetone) by measuring the loss of mass after being incubated in the solvents for different durations (6, 12, 24, 48, and 168 hours) at room temperature (22° C.). FIG. 5(g) depicts the thermal stability of the pollen-based substrate by measuring the change in length after being heated at different temperatures i.e., room temperature, 100° C., 200° C., and 250° C. for 30 minutes.

FIG. 6 includes snapshots of the process of folding the crane origami shown in FIG. 5(b) from the pollen-based substrate (lateral dimension: 8 cm×8 cm, thickness: 65 μm).

FIG. 7 includes SEM images (including magnified portions) and photographs of the pollen-based substrate after being dipped in various solvents (DI water, 2M NaOH, 2M HCl, and acetone) for 30 days.

FIG. 8 includes graphical illustrations of the change in resistance of an electrode formed on the pollen-based substrate via 3D printing, as well as substrates from PC, PI, and PET in (a) 2 M NaOH, (b) 2 M HCl, and (c) acetone for 7 days and also photographs of the substrates with the electrical disconnection corresponding to the asterisk (*) points in the graphical illustrations.

FIG. 9 includes (a) a FT-IR spectrum of the pollen-based substrate after being subjected to heat treatment at various temperatures (RT, 100° C., 200° C., and 300° C.) and (b) a thermogravimetric analysis result of the pollen-based substrate.

FIG. 10 is a schematic illustration of the process to fabricate a pollen substrate-based electrophysiological (EP) sensor, including the steps (a) fabricating the EP sensor and (b) transfer printing the EP sensor onto the pollen-based substrate.

FIG. 11(a) is a schematic illustration of the pollen substrate-based EP sensor. FIG. 11(b) and FIG. 11(c) are a photograph and a microscopy image respectively of the resulting pollen substrate-based EP sensor obtained from the fabrication process. FIG. 11(d) is a cross-sectional SEM image of the pollen substrate-based EP sensor, revealing the interfacial layers within the sensor. FIG. 11(e) includes a photograph and the corresponding electromyography (EMG) measurement results when the pollen substrate-based EP sensor is in contact with the forearm skin (compared to a conventional EP recording electrode). FIG. 11(f) includes a photograph and the corresponding electrocardiogrameasurement results when the pollen substrate-based EP sensor is in contact with the chest (compared to a conventional EP recording electrode).

FIG. 12 is a magnified view of an ECG signal recorded by (a) a conventional recording electrode and (b) the pollen substrate-based EP sensor.

FIG. 13(a) is a schematic illustration of the process to fabricate the stretchable pollen substrate-based heating patch. FIG. 13(b) includes photographs of the resulting pollen substrate-based heating patch obtained from the fabrication process—the magnified photographs are of the heating patch undergoing stretching at the applied strain of 0% (no strain) and 40%. FIG. 13(c) is a normalized resistance versus strain curve of the pollen substrate-based heating patch. FIG. 13(d) is a temperature profile curve of the pollen substrate-based heating patch at the applied voltages of 0.5 V, 0.75 V and 1 V. FIG. 13(e) includes photographs and the corresponding FEA results of the pollen substrate-based heating patch under stretching at an applied strain of 0% (no strain), 20% and 40%.

FIG. 14 includes micrograph images of the pollen-based substrate before and after laser cutting as well as the top, tilted, and cross-sectional SEM images of the pollen-based substrate at the cutting edge.

FIG. 15 includes the top, tilted, and cross-sectional SEM images and optical microscope images of the printed Ag flake/PU layer on the pollen-based substrate.

FIG. 16 includes graphical illustrations of the relative change in the resistance of the pollen-based substrate-based heating patch under repeated cycles of stretching at an applied strain of 30% and 40%.

FIG. 17 includes temperature profiles of the heating patch built on polyimide (PI), polyethylene terephthalate (PET) substrates as well as the pollen-based substrate at applied voltages of 0.50 V, 0.75 V, and 1.00 V.

FIG. 18(a) is a schematic diagram of the circuitry embedded in a custom-made wristwatch control unit connected to the pollen substrate-based heating patch FIG. 18(b) is a photograph of the pollen substrate-based heating patch connected to the wristwatch control unit on a wrist. FIG. 18(c) is an IR image of FIG. 18(b) when voltage of 1.7 V is applied to the pollen substrate-based heating patch. FIG. 18(d) is a temperature profile of the pollen substrate-based heating patch under repeated on/off cycles.

FIG. 19 includes photographs of the custom-made wristwatch control unit (a) with and (b) without a housing package.

### DESCRIPTION

It has been surprisingly found that pollen grains can provide a substrate material that is readily available, economical, biodegradable, and biocompatible that can be used as a replacement for synthetic substrate materials in electronics. This material can be used to create a variety of flexible shapes with customized mechanical, geometrical, electronic, and functional properties and performance characteristics such as thermal, chemical, and mechanical stability and optical transparency. Thus, in a first aspect of the invention, there is provided a conductive composite material, comprising:
a pollen-based substrate layer;
an elastomeric adhesive layer on top of the pollen-based substrate layer;
a biocompatible polymer substrate layer on top of the elastomeric adhesive layer; and
a metal layer on top of the biocompatible polymer substrate layer.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, and the like.

Pollen is primarily composed of structural proteins, lipids, and sporopollenin in the exine (outer layer), and cellulose, hemicellulose, and pectin in the intine (inner layer). Sporopollenin has high strength and can protect cellular materials from harsh environmental conditions such as high or low temperatures or exposure to acidic or alkaline solvents. Considering its practically indestructible structural characteristics, sporopollenin may be considered for several applications such as micromotors and drug delivery systems. Pollen is also less expensive than commercial polymers such as polylactic acid (PLA) and polyamide (PA).

The pollen-based substrate layer may be made from any suitable pollen-based material. For example, a suitable pollen-based substrate may be a material that comprises a plurality of pollen microgels.

The pollen microgels may be obtained from pollen grains from a suitable source (e.g. sunflower and/or commercial raw bee pollen). The process to make pollen microgels may involve defatting the pollen, which can then be mixed with a solution (e.g. 10% w/w) of KOH at an elevated temperature (e.g. about 80° C.) for a suitable period of time (e.g. about 5 hours) to produce soft pollen microgels. These soft pollen microgels can then be collected and washed, cast into a mold and then left to dry to produce a pollen-based substrate comprising the soft pollen microgels, which has a microscopically rough surface topography, owing to the intrinsic nano- and microstructures of the self-assembled pollen grains. As will be appreciated, the dimensions of the substrate are only limited by the casting mold used. It will be appreciated that any suitable pollen may be used to manufacture a pollen-based substrate (e.g. sunflower pollen).

As noted hereinbefore, the plurality of pollen grains may be derived from any suitable source. For example, the plurality of pollen microgels may be derived from pollen grains from one or more of the group including, but not limited to, sunflower (Helianthus annuus L.) pollen grains, pine (Pinus taeda) pollen grains, daisy (Baccharis halimifolia L.) pollen grains, cattail (Typhae angustfolia) pollen grains, camellia (Camellia Sinensis L.) pollen grains, bee pollen grains, and lycopodium (Lycopodium clavatum) spores (S-type).

The elastomeric adhesive layer may be any suitable adhesive material that can be used in a flexible and stretchable electronic applications. For example, the elastomeric adhesive layer on top of the pollen-based substrate layer is formed from a silicone elastomer and/or an acrylic adhesive. The silicone elastomer may be any suitable silicone elastomer. In more particular embodiments of the invention that may be mentioned herein, the elastomeric adhesive layer on top of the pollen-based substrate layer may be a platinum-cured silicone elastomer. Examples of suitable silicone elastomers include polydimethylsiloxane (PDMS), and platinum-cure silicones, such as those sold under the tradenames Silbione™, EcoFlex™ silicones, and Solaris™ silicones. Any suitable acrylic adhesive may be used herein and in certain embodiments the adhesive layer may be an acrylic adhesive.

The biocompatible polymer substrate layer on top of the elastomeric adhesive layer may be any suitable polymeric material. More particularly, it may be any suitable polymeric material that will not dissolve in any processing conditions applied during the manufacture of the conductive composite material discussed herein. In particular embodiments of the invention that may be mentioned herein, the biocompatible polymer substrate layer on top of the elastomeric adhesive layer may be formed from one or more of polyimide, polyethylene terephthalate (PET), a polyurethane, and a parylene. In more particular embodiments of the invention that may be discussed herein, the biocompatible polymer substrate layer on top of the elastomeric adhesive layer may be formed from polyimide

The metal layer on top of the biocompatible polymer substrate layer may be formed from any suitable metal or alloy. In particular embodiments of the invention that may be mentioned herein, the metal layer on top of the biocompatible polymer substrate layer may be formed from chromium (Cr), titanium (Ti), copper (Cu), platinum (Pt), silver (Ag), gold (Au), and alloys thereof.

In certain embodiments the metal layer may actually be in the form of two layers. That is, the metal layer on top of the biocompatible polymer substrate layer may be presented as a first layer and a second layer, with the first layer in direct contact with the biocompatible polymer substrate layer. The first layer may be formed from any suitable metal. For example, the metal in the first layer may be formed from one or more of chromium (Cr), titanium (Ti), and copper (Cu), and the second layer may be formed from one or more of platinum (Pt), silver (Ag), and gold (Au). In particular embodiments of the invention that may be mentioned herein, the first layer may be chromium (Cr) and the second layer may be gold (Au).

The metal layer (as a whole) may have any suitable thickness, provided that it is useful in flexible electronics. In embodiments where the metal layer is provided as a first and second layer, then the first layer may have a thickness of from 1 to 50 nm, such as from 2 to 25 nm, such as about 5 nm and the second layer may have a thickness of from 100 to 250 nm, such as from 125 to 200 nm, such as about 150 nm. As will be appreciated, the sum totals of these values may also provide a suitable thickness for the metal layer if it is formed from a single layer.

As noted herein, the conductive composite material may be useful for using in flexible electronics. With that in mind, it will be appreciated that the metal layers may be patterned to provide suitable circuitry and/or electrodes. For example, in certain embodiments of the invention that may be mentioned herein, the metal layer may be patterned to provide electrodes.

As discussed herein, the patterning process will generally take place after the after the deposition of the metal layer on the biocompatible polymer substrate layer. Given this, the biocompatible polymer substrate layer may also be patterned at the same time as the metal layer. Additionally or alternatively, the patterning may take place after assembly of the conductive composite material, so that all layers are patterned.

The conductive composite material disclosed herein may be used in electronic devices for health monitoring and wearable wireless heating, amongst other uses. Further applications include, but are not limited to wearable sensors, implantable devices, and soft robotics.

The pollen-based substrate layer disclosed herein can function as an ideal platform for a broad range of applications such as wearable biomedical devices, flexible/stretchable electronics, and soft robotics, and represents a low-cost and scalable solution to replace plastic substrates across electronic sensor applications.

The conductive composite material as disclosed herein may be formed by any suitable method. In a second aspect of the invention, there is provided a method of forming a conductive composite material as described herein, the method comprising the steps of:
(ai) providing a pollen-based substrate layer and a conductive intermediate comprising:
   a biocompatible polymer substrate layer; and
   a metal layer on top of the biocompatible polymer substrate layer; and
(aii) attaching the conductive intermediate to the pollen-based substrate layer by an elastomeric adhesive to provide the conductive composite material,
   optionally wherein the metal layer is patterned to provide an electrode.

The conductive intermediate in the method above may be provided by the steps of:
(bi) depositing a layer of polymethyl methacrylate (PMMA) on a substrate to provide a PMMA-coated substrate;
(bii) depositing a layer of a biocompatible polymer on the PMMA-coated substrate;
(biii) depositing a metal layer on the biocompatible polymer layer; and
(biv) immersing the PMMA layer in acetone to dissolve the PMMA layer and release the conductive intermediate.

As these methods provide the conductive composite material disclosed hereinbefore, a discussion of the same materials mentioned hereinbefore will be omitted for the purposes of brevity.

In a further aspect of the invention, there is provided a stretchable biopolymer-based heating pad, comprising:
a pollen-based substrate layer;
a cured polymeric layer or a metal layer on top of the pollen-based substrate layer, where, when present, the cured polymeric layer comprises a cured polymeric material and a metal; and
an encapsulation coating comprising a polymeric material that encapsulates the cured polymeric layer or the metal layer, wherein
the pollen-based substrate layer and the cured polymeric layer are patterned to provide a heating pad.

While this aspect of the invention is discussed herein in relation to a heating pad, it will be appreciated that this aspect may relate to any device or apparatus having the components mentioned above, which may have other alternative applications.

The encapsulation coating may in certain embodiments only encapsulate the cured polymeric layer or the metal layer. However, in other embodiments that may be mentioned herein the encapsulation coating may further encapsulate the pollen-based substrate layer.

When the cured polymeric layer or the metal layer on top of the pollen-based substrate layer is a metal layer it may be formed from any suitable metal. For example, the metal layer on top of the pollen-based substrate layer may be formed from one or more of the group consisting of chromium (Cr), titanium (Ti), copper (Cu), platinum (Pt), silver (Ag), and gold (Au). This material may be provided

As mentioned previously, the metal layer on top of the pollen-based substrate layer may be presented as a first layer and a second layer, with the first layer in direct contact with the pollen-based substrate layer. Given this, the first layer may be formed from one or more of chromium (Cr), titanium (Ti), and copper (Cu), and the second layer may be formed from one or more of platinum (Pt), silver (Ag), and gold (Au), optionally wherein the first layer is chromium (Cr) and the second layer is gold (Au). Additionally or alternatively, the first layer may have a thickness of from 1 to 50 nm, such as from 2 to 25 nm, such as about 5 nm and the second layer may have a thickness of from 100 to 250 nm, such as from 125 to 200 nm, such as about 150 nm.

The pollen-based substrate layer is identical to the pollen-based substrate layer described in relation to the conductive composite material in the first aspect of invention hereinbefore. As such, discussion of the pollen-based substrate layer to this aspect of invention is omitted for the sake of brevity.

When the cured polymeric layer or the metal layer on top of the pollen-based substrate layer is a cured polymeric layer, then it may be provided in the form of a cured polymeric material and a metal. For example, the metal may be distributed within the cured polymer in the form of metal flakes.

The cured polymeric material may be any suitable cured polymeric material for use in flexible electronics. For example, the cured polymeric material may be selected from one or more of a polyurethane and a silicone elastomer. In particular embodiments of the invention that may be discussed herein, the cured polymeric material may be a polyurethane.

The metal used in the metal in the cured polymeric material may be any suitable metal. For example, the metal in the cured polymeric material may be selected from one or more of the group consisting of platinum (Pt), silver (Ag), and gold (Au), optionally wherein the metal is silver (Ag). This metal may be in any suitable form that enables it to be distributed within the cured polymeric material. For example, the metal may be provided in the form of metal flakes.

As discussed below, the cured polymeric material and the metal may be printed onto the pollen-based substrate layer. As such, a formulation comprising an uncured polymeric material suitable for curing and a metal (e.g. metal flakes) may be used for the printing step.

As will be appreciated, the encapsulation coating may be any suitable material that can be used in a flexible and wearable electronic device. For example, the encapsulation coating may be a silicone elastomer. Examples of suitable silicone elastomers include polydimethylsiloxane (PDMS), and platinum-cure silicones, such as those sold under the tradenames Silbione™, EcoFlex™ silicones, and Solaris™ silicones. In particular embodiments of the invention, the encapsulation coating may be polydimethylsiloxane (PDMS).

In a further aspect of the invention, there is provided a method of forming a stretchable biopolymer-based heating pad as described herein, wherein the method comprises the steps of:
(ci) providing a heating pad intermediate comprising:
   a pollen-based substrate layer;
   a cured polymeric layer or a metal layer on top of the pollen-based substrate layer, where, when present, the cured polymeric layer comprises a cured polymeric material and a metal; and
(cii) encapsulating at least the cured polymeric layer or the metal layer on top of the pollen-based substrate layer with an encapsulation coating comprising a polymeric material.

The encapsulation coating is the same as described hereinbefore. Therefore, the encapsulation coating may be a silicone. More particularly, the encapsulation coating may be polydimethylsiloxane (PDMS).

In the method above, the encapsulating step (cii) further encapsulate the pollen-based substrate layer. That is, the encapsulating step may not only encapsulate the cured polymeric layer or a metal layer on top of the pollen-based substrate, but also the pollen-based substrate too.

The heating pad intermediate may be formed by the steps of:
(di) providing a patterned pollen-based substrate layer that has been patterned to provide the shape of a heating pad; and
(dii) applying a polymeric layer comprising a polymeric material and a metal onto the patterned pollen-based substrate layer and curing the polymeric layer after it has been applied or applying a metal layer onto the patterned pollen-based substrate layer.

In such embodiments, the patterned pollen-based substrate layer may be formed by:
(ei) providing a pollen-based substrate that has not been patterned; and
(eii) patterning the pollen-based substrate to provide the shape of a heating pad.

Further aspects and embodiments of the invention are described below.

The invention relates to a biopolymer/plant-based substrate for flexible and stretchable electronics and a method of fabricating the same. The biopolymer/plant-based substrate may be from plant pollen.

In a further aspect of the invention, there is provided a biopolymer substrate, comprising:
aa) a pollen-based layer;
ab) a thin layer of silicone elastomer (Silbione) on the pollen-based layer; and
ac) a Cr/Au layers/polyimide layer (electrode) on the silicone elastomer (Silbione).

In a further aspect of the invention, there is provided a stretchable biopolymer-based heating pad, comprising:
ba) a pollen-based layer; and
bb) a thin layer of silicone elastomer (Silbione) on the pollen-based layer;
bc) a Cr/Au layers/polyimide layer (electrode) on the silicone elastomer (Silbione);
bd) a cured Ag flake/PU ink layer on the Cr/Au layers/polyimide layer; and
be) an encapsulation layer of Polydimethylsiloxane (PDMS).

In a further aspect of the invention, there is provided a method to fabricate a biopolymer substrate, comprising the steps of:
ca) depositing a layer of poly(methyl methacrylate, PMMA) on a glass substrate;
cb) depositing a layer of polyimide on the PMMA-coated glass substrate;
cc) depositing layers of Cr (5 nm)/Au (150 nm) on the polyimide/PMMA-coated glass substrate;
cd) patterning the Cr/Au layers to form electrodes;
ce) immersing the substrate of (l) in acetone to dissolve the PMMA layer and release the remaining substrate; and
cf) adhering the obtained substrate of (m) onto a pollen-based substrate with a thin layer of a silicone elastomer (Silbione) sandwiched between these two substrates.

In a further aspect of the invention, there is provided a method to fabricate a stretchable biopolymer-based heating pad, comprising the steps of:
da) providing a biopolymer substrate according to the above;
db) patterning the biopolymer substrate;
dc) depositing Ag flake/PU ink onto the patterned biopolymer substrate to form conductive paths;
dd) curing the Ag flake/PU ink-coated biopolymer substrate; and
de) depositing a layer of PDMS onto the Ag flake/PU ink-coated biopolymer substrate to form an encapsulation layer.

In a further aspect of the invention, there is provided a biopolymer substrate, comprising:
ea) a pollen-based layer comprising pollen grains; pollen grains from the sunflower (Helianthus annuus L.), pine (Pinus taeda), and daisy (Baccharis halimifolia L.) families, lycopodium (Lycopodium clavatum) spores (S-type) and the cattail (Typhae angustfolia) pollen grains, camellia (Camellia Sinensis L.) bee pollen granules, etc;
eb) a thin layer of silicone elastomer/Biocompatible polymer or adhesives (for example Silbione, PDMS, EcoFlex, Solaris, Acrylic adhesives) on the pollen-based layer; and
ec) a metallic Cr/Au (can also be Titanium (Ti), Copper (Cu), Platinum (Pt), Silver (Ag)) layers/polyimide (examples of Biocompatible polymer substrate: PET, Polyurethane, PMMA, Perylene) layer (electrode) on the silicone elastomer/polymer (for example Silbione, PDMS, EcoFlex, Solaris, Acrylic adhesives).

In a further aspect of the invention, there is provided a stretchable biopolymer-based heating pad, comprising:
fa) a pollen-based layer comprising pollen grains; and pollen grains from the sunflower (Helianthus annuus L.), pine (Pinus taeda), and daisy (Baccharis halimifolia L.) families, lycopodium (Lycopodium clavatum) spores (S-type) and the cattail (Typhae angustfolia) pollen grains, camellia (Camellia Sinensis L.) bee pollen granules, etc.;
fb) a thin layer of silicone elastomer/polymer (for example Silbione, PDMS, EcoFlex, Acrylic adhesives) on the pollen-based layer;
fc) a metallic Cr/Au (can also be Titanium, Copper, Platinum, Silver) layers/polyimide (examples of Biocompatible polymer substrate: PET, Polyurethane, PMMA, Perylene) layer (electrode) on the silicone elastomer (for example Silbione, PDMS, EcoFlex, Solaris);
fd) a cured Ag (can also be Au, Cu, Pt) flake/PU (Styrene-ethylene-butylene-styrene; SEBS) ink layeron the metallic Cr/Au (can also be Titanium, Copper, Platinum, Silver) layers/polyimide (can also be PET, Polyurethane, PMMA, Perylene) layer; and
fe) an encapsulation layer of Polydimethylsiloxane (PDMS, can also be Silbione, EcoFlex, Solaris).

In a further aspect of the invention, there is provided a method to fabricate a biopolymer substrate, comprising the steps of:
ga) depositing a layer of poly(methyl methacrylate, PMMA) on a glass (Si wafer) substrate;
gb) depositing a layer of polyimide (can also be Parylene, PET) on the PMMA-coated glass substrate;
gc) depositing layers of Cr (5 nm)/Au (150 nm) (can also be Ti, Ag, Cu, Pt) on the polyimide/PMMAcoated glass substrate;
gd) patterning the metallic Cr/Au (can also be Ti, Ag, Cu, Pt) layers to form electrodes;
ge) immersing the substrate of (l) in acetone to dissolve the PMMA layer and release the remaining substrate; and
gf) adhering the obtained substrate of (m) onto a pollen-based substrate with a thin layer of a silicone elastomer (for example Silbione, PDMS, EcoFlex, Acrylic adhesives) sandwiched between these two substrates.

In a further aspect of the invention, there is provided a method to fabricate a stretchable biopolymer-based heating pad, comprising the steps of:
ha) providing a biopolymer substrate according to the above;
hb) patterning the biopolymer substrate;
hc) depositing Ag (can also be Au, Cu, Pt) flake/PU (SEBS) ink onto the patterned biopolymer substrate to form conductive paths;
hd) curing the Ag flake/PU ink-coated (q) biopolymer substrate; and
he) depositing a layer of PDMS (for example Silbione, EcoFlex, Solaris) onto the Ag flake/PU inkcoated biopolymer substrate to form an encapsulation layer.

The present disclosure provides a pollen-based substrate and its use in flexible and stretchable electronics. The pollen-based substrate enabled compelling advantages over conventional synthetic plastics in terms of natural sourcing, environmental degradation, and sustainable processing. Various analyses were performed to characterize the pollen-based substrate in terms of its physical, chemical, and thermal stability under appropriate practical conditions of intended applications. As proofs of concept, the present disclosure provides a skin-mountable stretchable EP sensor and heating patch based on the pollen-based substrate by using indirect and direct fabrication approaches. These embodiments are only examples, and are not intended to limit the scope, applicability of the present disclosure. The pollen-based substrate can function as an ideal platform for a broad range of applications such as wearable biomedical devices, flexible/stretchable electronics, and soft robotics, and represents a low-cost and scalable solution to replace plastic substrates across electronic sensor applications.

Further aspects and embodiments of the invention will now be discussed by reference to the following non-limiting examples.

### EXAMPLES

Further aspects and embodiments of the invention will be discussed by reference to the following non-limiting examples.

### Materials and Methods

### FT-IR Spectroscopy and TGA Measurement

Chemical analysis was conducted via Fourier-transform infrared (FT-IR) spectroscopy (Frontier, PerkinElmer) with a diamond cell attenuated total reflection accessory module. Transmittance was obtained using an ultraviolet-visible (UV-Vis) spectrometer (UV-2700, SHIMADZU) and thermal analysis was conducted using a Q500 instrument (TA Instruments).

### Sample Imaging

Optical micrograph and SEM images were obtained using a Nikon TS100 and a JSM-7600F Schottky field-emission scanning electron microscope (JEOL), respectively. A Park XE-100 device was used to obtain atomic force microscopy (AFM) images (non-contact mode, scan area=20 μm×20 μm, scan rate=0.2-0.5 Hz, NX-10, Park Systems) at RT by using an aluminum reflex-coated silicon cantilever, PPP-NCHR (spring constant≈42 N/m, Nanosensors).

Young's modulus measurement by AFM

To measure the Young's modulus through the AFM technique, a gold reflex-coated silicon cantilever, biosphere B2000-NCH (diameter=4 μm, spring constant≈40 N/m, Nanotools, Germany), was used to obtain the force-displacement (FD) curve from 16 points of the selected area in the pollen-based substrate (lateral dimension=10 μm×10 μm, approach speed=0.8 μm/s, and maximum loading force=6.0 uN). To remove the contaminants, the AFM cantilever was rinsed with water and ethanol, followed by UV cleaning for 30 min. To calculate Young's modulus, the Hertzian model was used for curve fitting in XEI (Park Systems) and Python. We determined Young's modulus from the FD curve according to the equation

$$F = \frac{E}{1 - {v}^{2}} [ \frac{{a}^{2} + {R}^{2}}{2} ln \frac{R + a}{R - a} - aR ] , δ = \frac{a}{2} ln \frac{R + a}{R - a}$$

where E is Young's modulus, v is Poisson's ratio, a is the contact radius, R is the radius of the sphere, and δ is the indentation depth. v was set as 0.5, which is a typical value for biological materials.

### Mechanical Tensile Test

A tensile test was performed using electromechanical universal test systems (MTS 42, MTS Systems Corporation), and the tensile speed was 1 cm/min at RT. The sample was prepared using a dumbbell-shaped cutter (dimension of the center=7.6 mm×3.2 mm, SDL-100, DUMBBELL CO., LTD.). The thickness and weight were measured using a micrometer (MDC-25PX, Mitutoyo Asia Pacific Pte Ltd.) and analytical weighing balance (BBX 22, BOECO, Germany), respectively. Moreover, a resistance-strain test was conducted using a dynamic mechanical analyzer (ESM303, Mark-10 Corp.) and source measure unit (Keithley 2400, Tektronix, Inc.). The temperature was measured using an IR camera (SC645, FLIR Systems, Inc.).

Recording of EP Signals from the Skin

The EP sensor was attached to the forearm skin and chest of a volunteer (age: 30 y) to capture the EMG and ECG signals, respectively. The generated EP signals were recorded using a preamplifier and data acquisition unit (Octal Bio Amp and Power Lab, ADInstruments) and transmitted to an external computing system. A commercial software (LabChart, ADInstruments) was used to process the collected data with a 60 Hz notch filter with a bandpass filter admitting frequencies of 10-500 Hz and 0.5-100 Hz for the EMG and ECG signals, respectively. The conventional EP recording electrodes were RedDot™ (3M).

### FEA

The deformations of the pollen-based substrate, Ag flake/PU, and PDMS were modeled by linear elastic behavior with the effective mechanical modulus (E) being 410 MPa, 60.8 GPa, and 1.84 MPa, respectively. The rule of mixtures was used to calculate the elastic modulus of Ag flake/PU ink (80% Ag and 20% PU). The entities were modeled using eight-node brick element with reduced integration (C3D8R) solid elements. Displacement boundary conditions were applied to both edges of the structure to produce uniaxial tension for strains of 10%, 20%, 30%, and 40%.

### Example 1—Preparation of the Pollen-Based Substrate

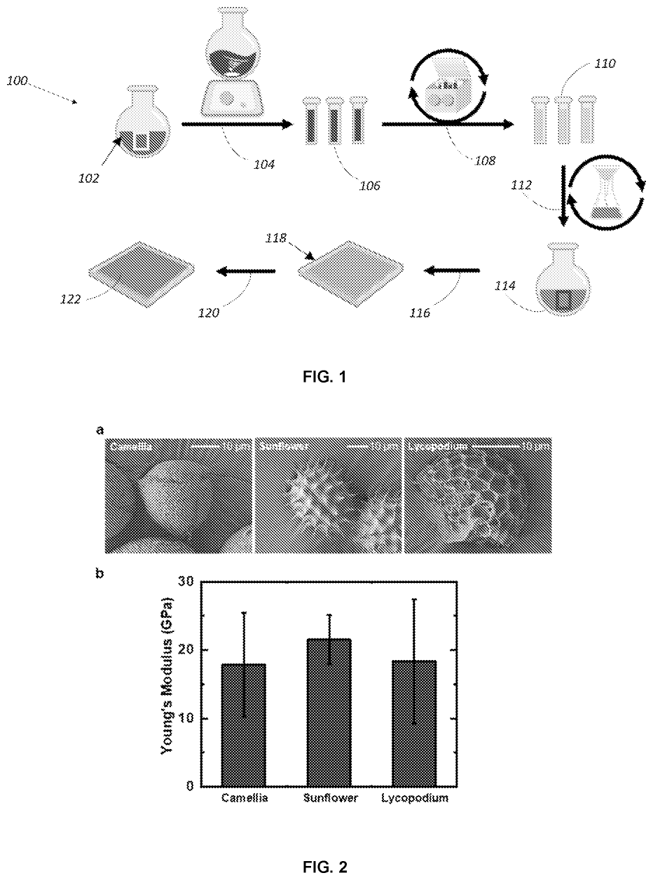

To produce the defatted Camellia pollen particles, 250 g of natural bee pollen granules (Xi'an Yuenum Biological Technology Company Ltd., Shannxi Xian, China) were refluxed with 500 mL of acetone (Sigma-Aldrich) in a round-bottom flask (1 L) under stirring (200 rpm, IKA® RCT) at 50° C. for 3 h. After stirring the pollen granules at 50° C. for 1 h with 1 L of deionized (DI) water (Direct-Q® Water Purification System, Merck), the pollen solution was sequentially filtered using a nylon mesh (ELKO Filtering Co., diameter=6 μm) and filter paper (diameter=6 μm, Whatman) to remove the contaminant. The pollen particles were refluxed in 500 ml of acetone while stirring at 50° C. for 3 h. Subsequently, the particles were subjected to vacuum filtration and dried for 12 h in a fume hood to remove acetone residues. Next, 20 g of dried pollen powder was refluxed with 250 mL of diethyl ether (Sigma-Aldrich) while stirring at 25° C. for 2 h, followed by two cycles of vacuum filtration to remove the low-polarity fat compound. Finally, the pollen solution, after stirring with diethyl ether at 25° C. for 12 h, was filtrated and dried for 12 h in a fume hood. FIG. 1 illustrates this process 100 schematically to fabricate the pollen-based substrate 122.

To remove the cytoplasm, the defatted pollen particles were suspended in 200 ml of potassium hydroxide (KOH) solution (Sigma-Aldrich, 10 wt % in DI water) to form a suspension 102. The suspension 102 was refluxed while stirring at 80° C. for 2 h in a round-bottom flask in a step 104. The suspension was then transferred to a conical tube 106 and centrifuged at 4500 rpm for 5 min (Allegra X-15R, Beckman Coulter, Inc.) to remove the KOH solution. The pollen particles were resuspended in 40 mL of fresh KOH and the suspension was centrifuged again in a washing step 108. The KOH washing step 108 was repeated for five times. After the fifth KOH washing step 108, the supernatant was discarded from the suspension 110 and the pollen particles were collected in a beaker (1 L) with 300 mL of DI water. The suspension was incubated while stirring at 50° C. for 5 min. The suspension was then filtered using a nylon mesh (pore size=10 μm) in a step 112. The filtration step 112 was repeated five times to neutralize the pollen solution 114 to a pH level of 7-7.5. After the fifth filtration step 112, the pollen solution 114 was cast into a mold 118 in a step 116 and then left to dry to produce a pollen-based substrate 122 comprising the soft pollen microgel.

Camellia pollen was selected to decrease the surface roughness of the substrate, as this pollen's surface was found to be smoother than that of other plants such as sunflower (Helianthus annuus L.) and lycopodium (Lycopodium clavatum) as observed in the SEM images in FIG. 2(a). Furthermore, the Young's modulus of the Camellia pollen (E=17.8 GPa) was measured and found to be similar to that of the sunflower pollen (21.5 GPa) and lycopodium spore (18.3 GPa) after defatting as shown in FIG. 2(b).

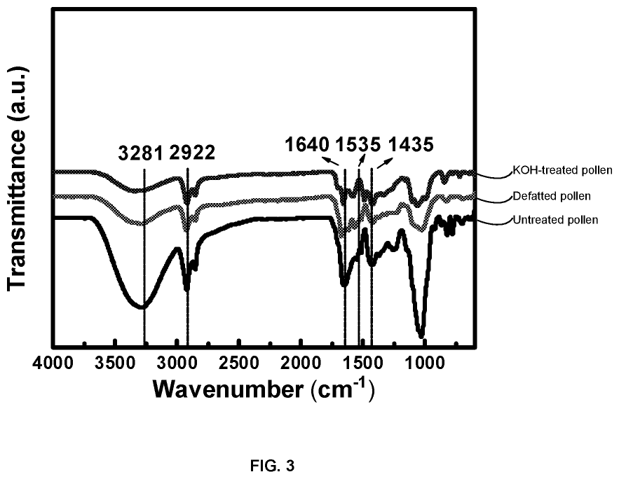

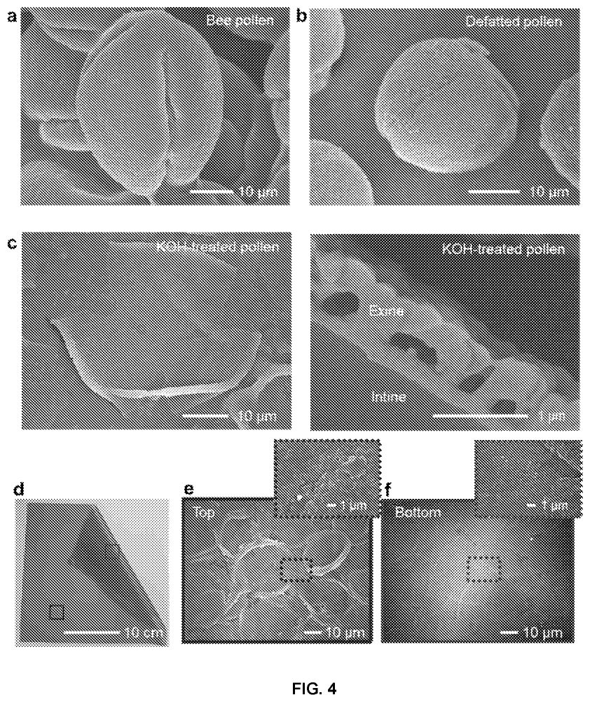

The lipids and proteins that cause allergic reactions to pollen were removed during defatting and subsequent treatment with KOH, as indicated by the Fourier-transform infrared (FT-IR) spectroscopic analysis result in FIG. 3. The peaks at 1435 cm⁻¹ (CH₂ deformation) corresponding to lipids and the peaks at 1640 cm⁻¹ (C═O stretching in amide I) and 1535 cm⁻¹ (N—H bending and C—N stretching in amide II) corresponding to proteins decreased after defatting and KOH treatment.

FIGS. 4(a), (b) and (c) are SEM images depicting the morphology of the pollen grains during the substrate fabrication process (i.e., (a) untreated (b) after being defatted and (c) after being treated with KOH). As seen in FIG. 4(b), the defatted sporopollenin particles were spherical with apertures and pores, and the particles enclosed the generative cell. In FIG. 4(c), although the KOH treated sporopollenin particle was flattened and hollow, it retained its strength after drying, and the unique structures of the exine and intine layers were visible.

FIG. 4(d) is a photograph of the resulting pollen-based substrate obtained from the fabrication process. FIGS. 4(e) and (f) are SEM images of the top and bottom sides of the pollen-based substrate of FIG. 4(d) respectively. The capillary forces induced by water evaporation both outside (i.e., between the pollen particles) and inside a pollen particle (i.e., between intines) facilitated the collapse and flattening of the sporopollenin layer. As the pollen particles underwent concomitant structural flattening, the carboxyl and hydroxyl functional groups on the surface of pollen components such as cellulose, pectate, and sporopollenin generated attractive forces, arising from van der Waals forces, electrostatic forces, and hydrogen bonding, to preserve the collapsed shape of pollen particles with tight particle-particle coupling.

### Example 2—Characterization of the Pollen-Based Substrate

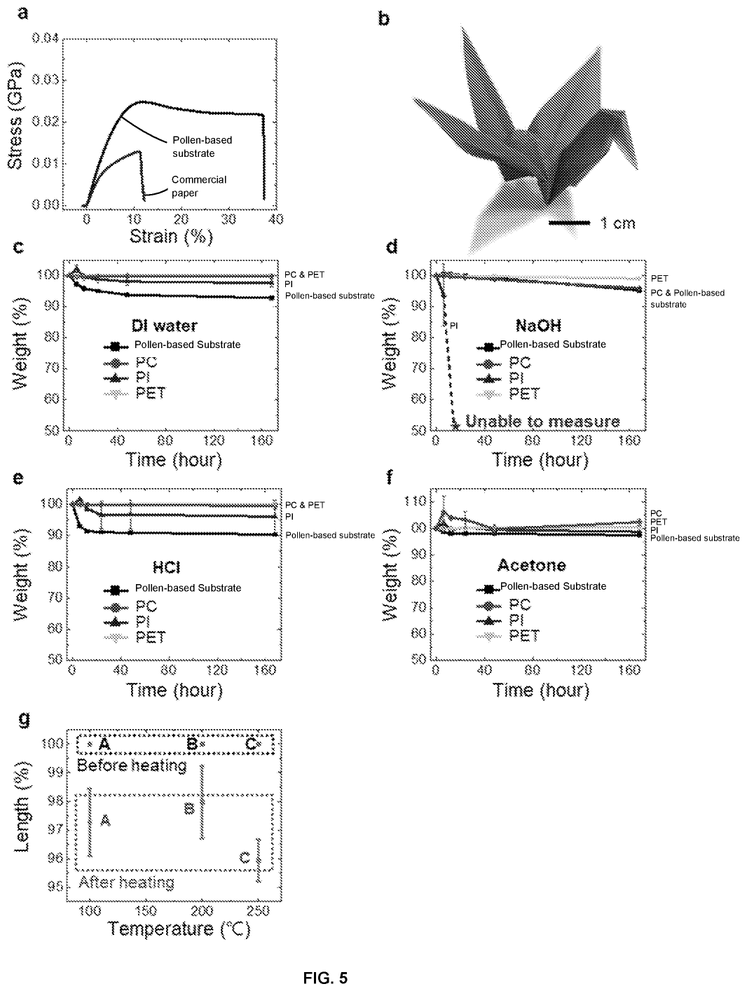

FIG. 5(a), (c) to (g) show the characterization results of the pollen-based substrate obtained from the fabrication process in Example 1.

FIG. 5(a) is a strain-stress curve of the pollen-based substrate compared to a commercial A4 paper (PaperOne™, April Group). The Young's modulus and tensile fracture strain were estimated to be 410 MPa and 37%, respectively, based on a uniaxial tensile test performed on the pollen-based substrate sheet. For the commercial A4 paper (PaperOne™, April Group), the corresponding values were 300 MPa and 12%, respectively.

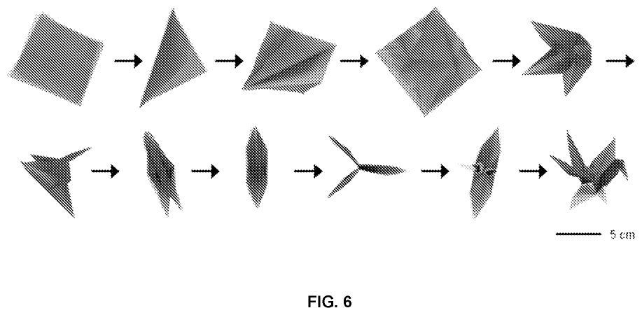

To illustrate the deformability of the pollen-based substrate and its amenability to being folded into complex shapes without cracking, an origami arrangement in the shape of a crane was made as shown in FIG. 5b and according to FIG. 6. The crane origami with 24 lines of inward folding, 30 lines of outward folding, and 24 intersection points of the folding lines did not exhibit any notable cracking.

FIGS. 5(c), (d), (e) and (f) depict the higher chemical resistance of the pollen-based substrate to various solvents, such as deionized (DI) water, 2 M NaOH, 2 M HCl, and acetone, as compared to polycarbonate (PC), polyethylene terephthalate (PET), and polyimide (PI) films that are commonly used as substrates for flexible electronics. According to mass measurements, the pollen-based substrate exhibited a mass loss of 7.3%, 4.8%, 9.7%, and 2.7% in DI water, NaOH, HCl, and acetone after 7 days. On the other hand, the PI film fully dissolved in NaOH after only 12 hours, and the PC film became brittle and opaque after being exposed to acetone for 6 hours.

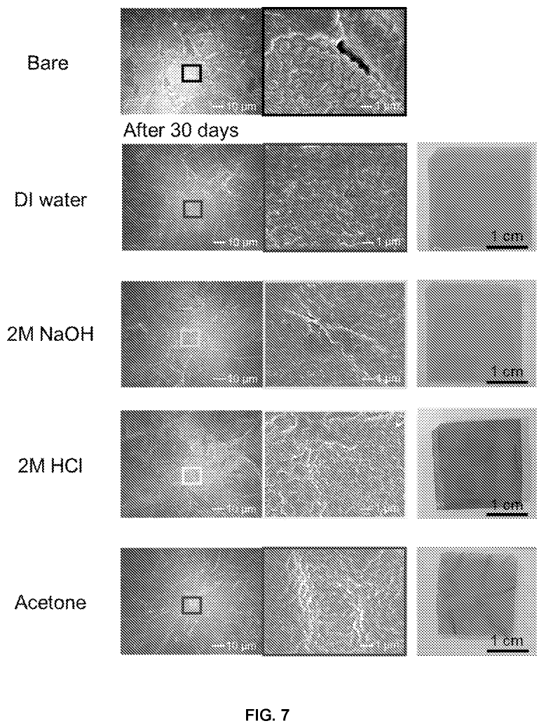

FIG. 7 includes SEM images (including magnified portions) and photographs of the pollen-based substrate after being dipped in various solvents (DI water, 2M NaOH, 2M HCl, and acetone) for 30 days. As shown, there was no significant structural distortion even after 30 days in all of the tested solvents.

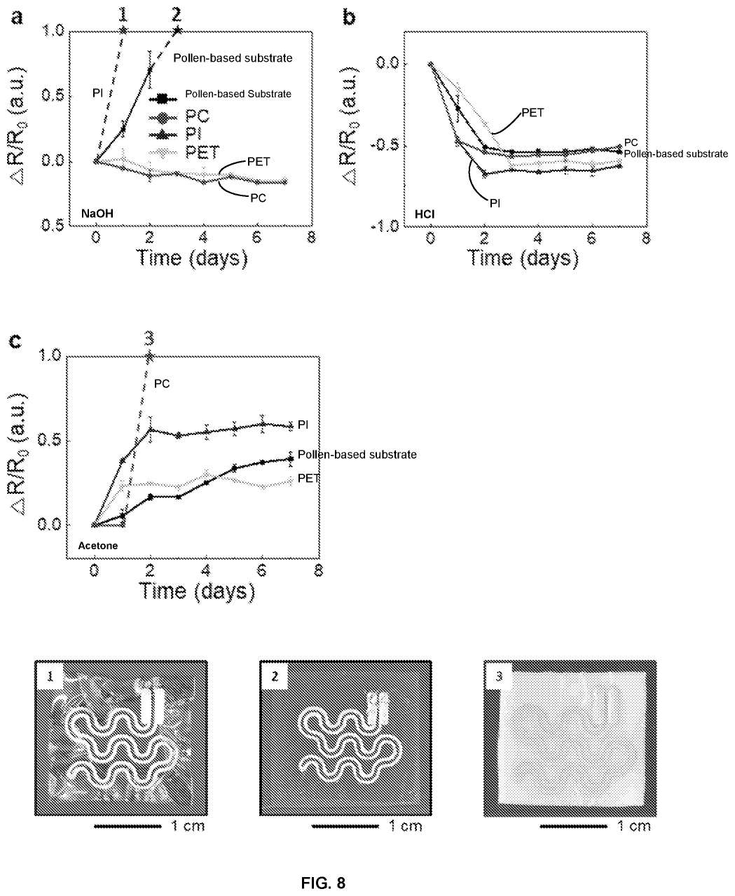

To confirm the potential for electronic applications using the pollen-based substrate material, an electrode was formed on each of the pollen-based substrate and comparison substrates i.e., polycarbonate (PC), polyimide (PI) and polyethylene terephthalate (PET) via 3D printing. As shown in FIG. 8, in the case of the PI and PC substrates, the electrode was disconnected within 1 day due to the dissolution and shape distortion of the substrates. Although the pollen-based substrate showed a high resistance in NaOH after 2 days, it was caused by delamination of the electrode from the substrate.

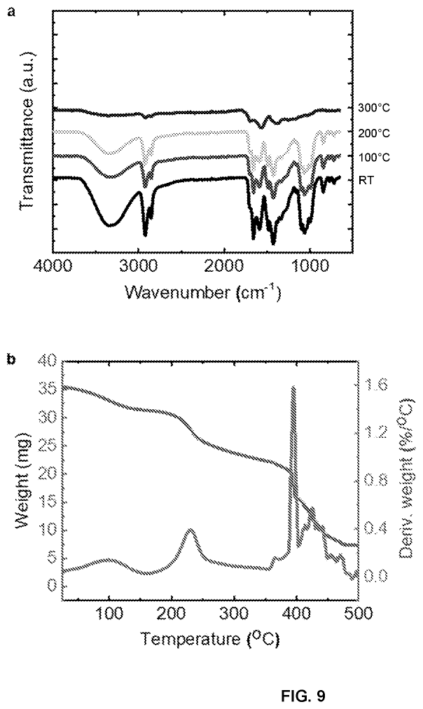

To evaluate the thermal stability of the pollen-based substrate, the dimensions were measured after heating at different temperatures: room temperature (RT), 100° C., 200° C., and 250° C. for 30 minutes. As shown in FIG. 5(g), the decrease in the size of the pollen-based substrate was small (3%) at 100° C. and 200° C. Furthermore, the chemical structure did not change significantly, as indicated by the FT-IR analysis result shown in FIG. 9(a). The thermogravimetric analysis (TGA) result shown in FIG. 9(b) also indicated that negligible weight change occurred at the temperature ranging from 110° C. to 210° C. These findings indicate that the pollen-based substrate is stable up to a temperature of 210° C. The pollen-based substrate, when heated to ≥250° C., exhibited a sharp decrease in weight although a dimensional change of only 4% occurred due to water evaporation from the constituent sporopollenin biopolymer. The results suggest that the pollen-based substrate can maintain its structural properties, shape, and chemical composition, even at operating temperatures that exceed the typical service conditions for electronic devices.

To demonstrate the suitability of the pollen-based substrate for flexible electronics, a pollen-based electrophysiological (EP) sensor and a stretchable pollen-based heating pad are provided by adopting two widely used fabrication methods: indirect (transfer printing) and direct (patterning) methods.

### Example 3 Fabrication of Pollen-Substrate Based Electrophysiological (EP) Sensor

First, an electrophysiological (EP) sensor, consisting of reference, ground, and recording electrodes, was fabricated on the pollen-based substrate through transfer printing.

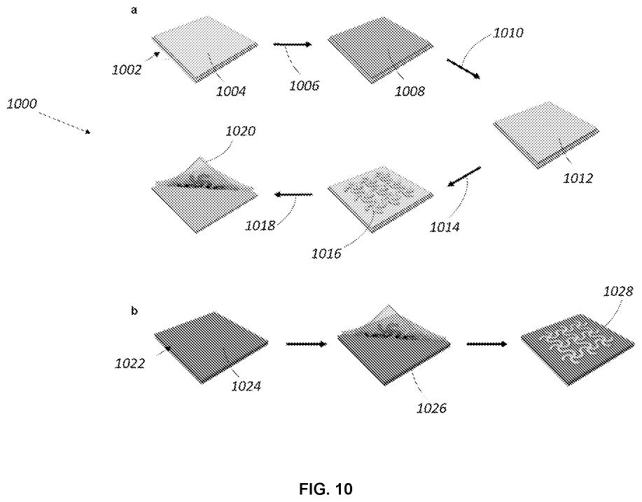

FIG. 10 is a schematic illustration of the process 1000 to fabricate the pollen substrate-based EP sensor 1028. A layer of polymethyl methacrylate (PMMA, 1 μm thick) 1004 was spin coated on a glass surface 1002. A successive layer of PI (1 μm thick) 1008 was spin-coated next in a step 1006. Electron beam deposition was performed in a step 1010 to deposit metal layers of chromium (Cr, 5 nm thick) and gold (Au, 150 nm thick) 1012. A combination of steps 1014 including standard photolithographic patterning with a photoresist (AZ 1518, 3000 rpm, 30 s), wet etching with Cr and Au etchant (Transene, Inc.) and oxygen plasma reactive-ion etching were performed to define a stretchable form (i.e., filamentary serpentine mesh) 1016 for the EP electrodes. The resulting structure was immersed in acetone in a step 1018 to dissolve the bottom PMMA layer and to allow the thin film metal layer 1020 to be released from the glass 1002. The released thin film metal layer 1020 was then transferred onto the pre-formed pollen-based substrate 1022 by using a thin layer (<10 μm thick) of silicone adhesive (Silbione, RT GEL 4317, Elkem Silicones) 1024. Specifically, the thin film metal layer 1020 was attached to the silicone adhesive 1024 on the pollen-based substrate 1022 in a step 1026 to form the pollen substrate-based EP sensor 1028.

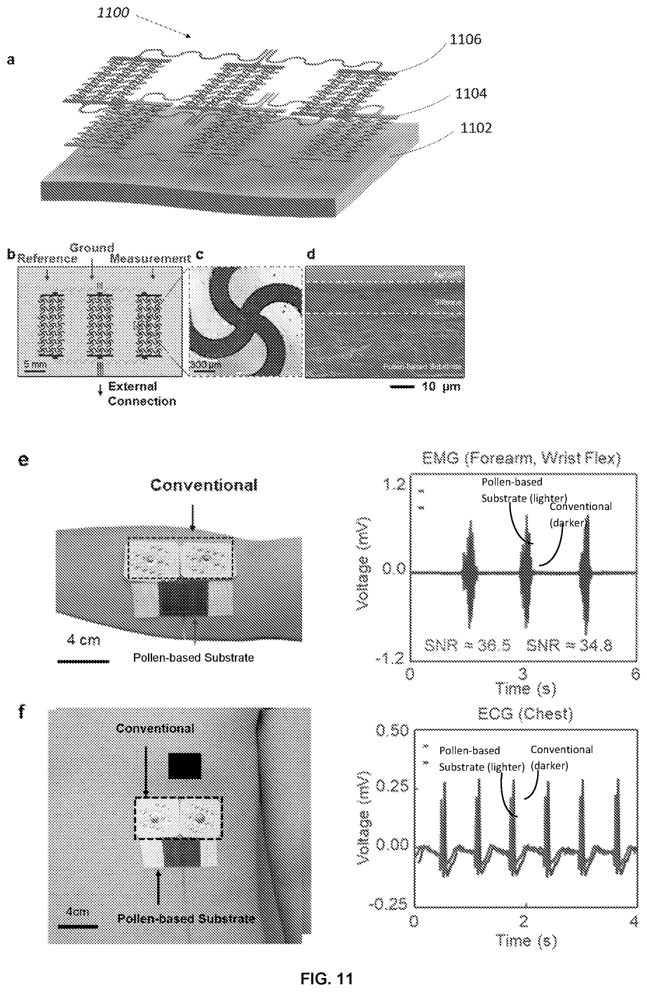

For reference, FIG. 11a is a schematic illustration of the pollen substrate-based EP sensor 1100 comprising the pollen-based substrate (100 μm) 1102, the polyimide layer (1 μm) 1104 and the Au electrode layer (150 nm) 1106. FIGS. 11(b) and (c) are a photograph and a microscopy image respectively of the resulting pollen substrate-based EP sensor obtained from the fabrication process. FIG. 11(d) is a cross-sectional SEM image of the pollen-substrate based EP sensor, revealing the interfacial layers within the sensor.

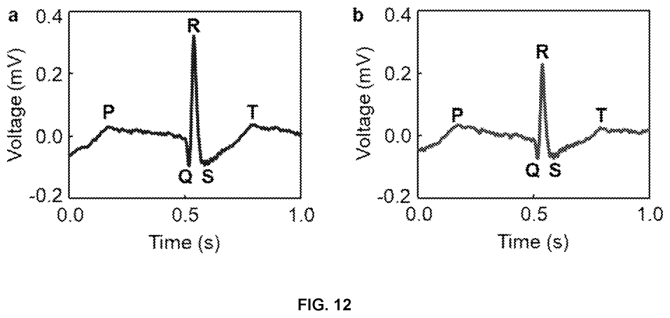

High-fidelity recordings of electromyogram (EMG) and electrocardiogram (ECG) signals were obtained by attaching the fabricated pollen substrate-based EP sensor on the right forearm and chest of a healthy adult as shown in FIGS. 11(e) and (f) respectively. The results were similar to those simultaneously obtained using conventional EP recording electrodes. Moreover, no skin irritation or discomfort was noticed following>30 minutes of the use. The EMG signals acquired during three consecutive wrist flexes exhibited comparable signal-to-noise ratios (SNR) (pollen-based substrate: 36.5, conventional: 34.8), baseline noise levels (pollen-based substrate: 1.79 μV, conventional: 1.92 μV), and a Pearson correlation coefficient (r)—which indicates the statistical association between the two signals—of 85%. FIG. 12 is a magnified view of the ECG signal recorded from (a) conventional EP recording electrode and (b) pollen substrate-based sensor. Notably, the ECG signals exhibited a high r value of ˜98% and were indicative of a P-wave (atrial depolarization), a QRS-complex (ventricular depolarization), and a T-wave (ventricular repolarization).

### Example 4 Fabrication of Stretchable Pollen Substrate-Based Heating Patch

Next, a stretchable pollen substrate-based heating patch was fabricated through direct (patterning) printing.

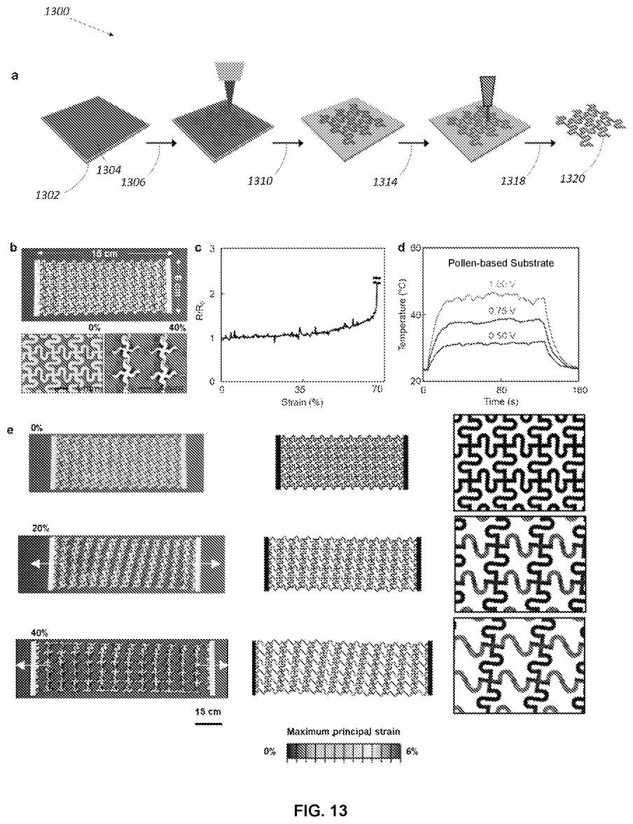

FIG. 13(a) is a schematic illustration of a process 1300 to fabricate a skin-mountable stretchable pollen-substrate based heating patch 1320. First, a pre-formed pollen-based substrate 1304 was mounted on a glass substrate 1302. A high-precision laser cutter (PLS6MW, Universal Laser systems, 15 W CO₂ laser, λ=10.6 μm, spot size=130 μm and spatial resolution=25.4 μm) was used to pattern an outline of the Joule heating element in a step 1306. The excess regions of the pollen-based substrate not required in the Joule heating element were removed in a step 1310. Conduction paths were printed on the surface on the pollen-based substrate by extruding the Ag flake/PU ink by using a three-axis automated fluid dispensing robot (PRO4, Nordson EFD) and digital pneumatic regulator (Ultimus V High-Precision Dispenser, Nordson EFD) in a step 1314.

The Ag flake/PU ink was prepared by first dissolving 1.48 g of PU (Elastollan Soft 35A, BASF) in a solution of 1.56 mL of N,N-dimethylformamide (DMF) (Sigma-Aldrich) and 4.27 ml of tetrahydrofuran (THF) (Sigma-Aldrich). Next, 5.92 g of Ag flakes (99.95%, average particle size (APS) 2-5 μm; 47MR-10F, Inframat Advanced Materials) was mixed in a planetary mixer (ARE-310, Thinky U.S.A., Inc.) for 10 min at 2000 rpm. The resulting mixture was loaded into a 3 cc syringe (Nordson EFD) and centrifuged at 2500 rpm for 2 min to remove the air bubbles trapped in the mixture.

The printed Ag flake/PU layer was cured in a vacuum oven at 50° C. for 4 h, followed by automated dispensing of PDMS to form an encapsulation layer in a step 1318 to form the pollen-substrate based heating patch 1320. The external power source was a DP832 device (Rigol).

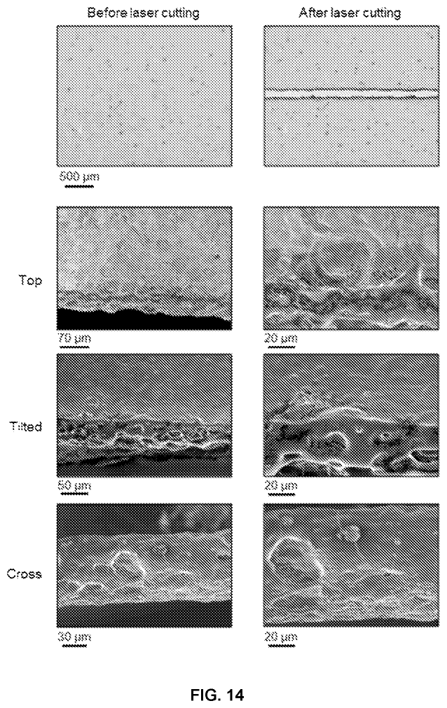

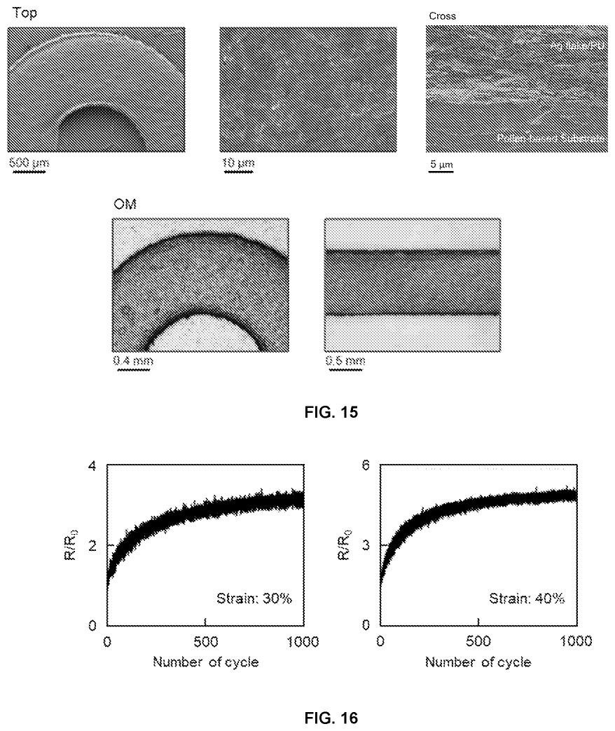

For reference, the pollen-based substrate was successfully patterned into a stretchable Joule heating configuration using a laser cutter that induce no thermal damage or distortion, as indicated in the optical images in FIG. 14. In addition, the Ag flake/PU ink and polydimethylsiloxane (PDMS) were directly printed onto the pollen-based substrate as indicated in FIG. 15. FIG. 13(b) shows photographs of the resulting pollen substrate-based heating patch obtained from the fabrication process—the magnified photographs are of the heating patch undergoing stretching at the applied strain of 0% (no strain) and 40%.

To characterize the resulting pollen substrate-based heating patch, the relative change in the resistance which is the ratio of the instantaneous resistance to the resistance at zero strain, R/R₀, of the heating patch was measured at uniaxial stretching of up to 65%. The result is shown in FIG. 13(c). The R/R₀ remained constant (less than 1.5) until an elongation strain of up to ˜40% and then abruptly increased until it reached the limit at ˜65%. The R/R₀ remained at 3 and 5 for more than 1,000 cycles of stretching at the applied strain of up to 30% and 40%, respectively as shown in FIG. 16.

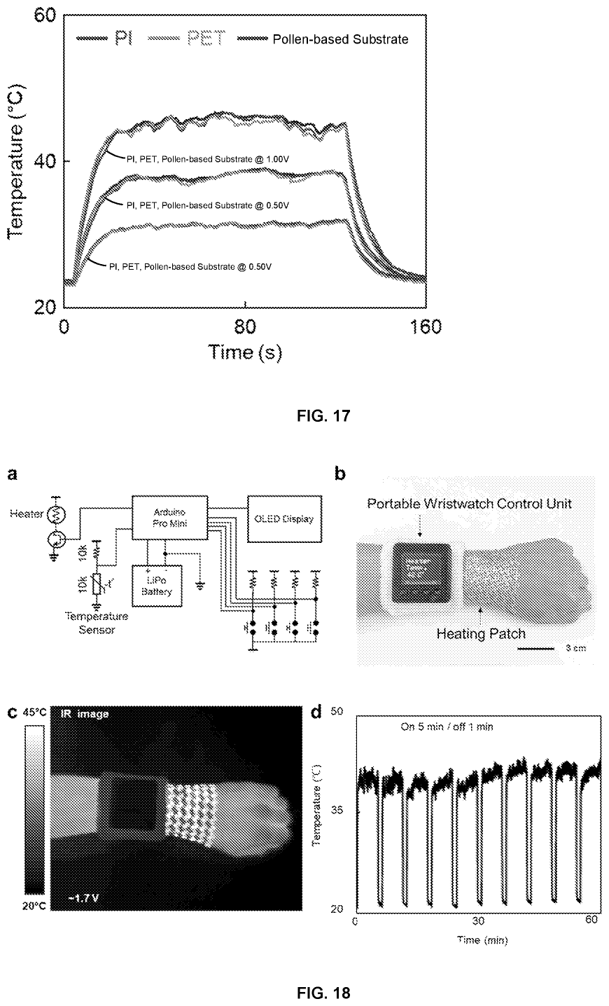

FIG. 13(d) shows the experimental results obtained from applying input voltages of 0.5 V, 0.75 V, and 1.0 V on the pollen substrate-based heating patch (3 cm×2 cm) for 120 seconds each with an external power source to reach the target temperature at approximately 31° C., 38° C., and 45° C., respectively. For comparison, the same heating patch was also printed on PI (80 μm thick) and PET (65 μm thick) substrates, which showed negligible differences in the target temperature (i.e. <0.67° C.) as shown in FIG. 17.

FIG. 13(e) shows photographs and the corresponding finite element analysis (FEA) results of the pollen substrate-based heating patch under stretching at the applied strain of 0% (no strain), 20% and 40%. The results of finite element analysis (FEA) modeling complemented these findings and indicated local maximum principal strains (εₘₐₓ) of <6% across the entire surface of the heating patch under stretching at the applied strain up to 40%.

Articular thermotherapy is often practiced to alleviate joint pain and stiffness by inducing vasodilation to promote blood flow to the target area. To achieve therapeutic results, conformal contact with the skin must be ensured to realize efficient heat transfer and precise temperature control (˜ 40° C.). To demonstrate this, the above pollen substrate-based heating patch was applied on the wrist of a human subject and the results discussed hereon.

FIG. 18(b) is a photograph of the pollen substrate-based heating patch connected to a wristwatch control unit on the wrist. The custom-made portable wristwatch was wired to the heating patch as a control unit that could maintain the temperature at less than 45° C. to avoid any risk of skin burn. FIG. 18(a) is a schematic diagram of the circuitry embedded in the wristwatch control unit. The wristwatch included the following key components: (1) a 10 kΩ thermistor for temperature measurement, (2) an organic light-emitting diode (OLED) to display the measured temperature, (3) a microcontroller for the control unit, (4) button switches for user controls (i.e., display/temperature settings and power on/off functions), and (5) a lithium-ion battery as the power source.

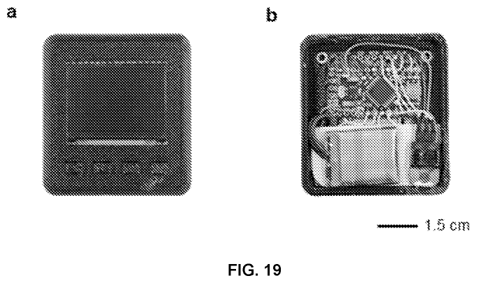

For reference, FIG. 19 shows photographs of the custom-made wristwatch control unit (a) with and (b) without a housing package. FIG. 18(c) shows a representative infrared (IR) image of the heating patch that was controlled by the wristwatch at the set temperature of 42° C. The wristwatch was also used to apply a pre-programmed heating cycle (e.g., 5 minutes on and 1 minute off) for slightly under 1 hour at the applied voltage of 1.0 V as shown in FIG. 18(d).

1. A conductive composite material, comprising:
a pollen-based substrate layer;
an elastomeric adhesive layer on top of the pollen-based substrate layer;
a biocompatible polymer substrate layer on top of the elastomeric adhesive layer; and
a metal layer on top of the biocompatible polymer substrate layer.

2. The conductive composite material according to claim 1, wherein the pollen-based substrate comprises a plurality of pollen microgels.

3. The conductive composite material according to claim 2, wherein the plurality of pollen microgels are derived from pollen grains from one or more of the group selected from sunflower (Helianthus annuus L.) pollen grains, pine (Pinus taeda) pollen grains, daisy (Baccharis halimifolia L.) pollen grains, cattail (Typhae angustfolia) pollen grains, camellia (Camellia Sinensis L.) pollen grains, bee pollen grains, and lycopodium (Lycopodium clavatum) spores (S-type).

4. The conductive composite material according to claim 1, wherein the elastomeric adhesive layer on top of the pollen-based substrate layer is formed from a silicone elastomer or an acrylic adhesive.

5. The conductive composite material according to claim 4, wherein the elastomeric adhesive layer on top of the pollen-based substrate layer is a platinum-cured silicone elastomer.

6. The conductive composite material according to claim 1, wherein the biocompatible polymer substrate layer on top of the elastomeric adhesive layer is formed from one or more of polyimide, polyethylene terephthalate (PET), a polyurethane, and a parylene.

7. (canceled)

8. The conductive composite material according to claim 1, wherein the metal layer on top of the biocompatible polymer substrate layer is formed from one or more of the group consisting of chromium (Cr), titanium (Ti), copper (Cu), platinum (Pt), silver (Ag), and gold (Au).

9. The conductive composite material according to claim 8, wherein the metal layer on top of the biocompatible polymer substrate layer is presented as a first layer and a second layer, with the first layer in direct contact with the biocompatible polymer substrate layer.

10. The conductive composite material according to claim 9, wherein the first layer is formed from one or more of chromium (Cr), titanium (Ti), and copper (Cu), and the second layer is formed from one or more of platinum (Pt), silver (Ag), and gold (Au).

11. (canceled)

12. The conductive composite material according to claim 1, wherein the metal layer is patterned to provide electrodes.

13. (canceled)

14. A method of forming a conductive composite material as described in claim 1, the method comprising the steps of:
(ai) providing a pollen-based substrate layer and a conductive intermediate comprising:
a biocompatible polymer substrate layer; and
a metal layer on top of the biocompatible polymer substrate layer; and
(aii) attaching the conductive intermediate to the pollen-based substrate layer by an elastomeric adhesive to provide the conductive composite material.

15. The method according to claim 14, wherein the conductive intermediate is provided by the steps of:
(bi) depositing a layer of polymethyl methacrylate (PMMA) on a substrate to provide a PMMA-coated substrate;
(bii) depositing a layer of a biocompatible polymer on the PMMA-coated substrate;
(biii) depositing a metal layer on the biocompatible polymer layer; and
(biv) immersing the PMMA layer in acetone to dissolve the PMMA layer and release the conductive intermediate.

16. The method according to claim 15, wherein the metal layer if formed from a first metal layer and a second metal layer, where the first metal layer is formed from one or more of chromium (Cr), Titanium (Ti), and Copper (Cu), and the second metal layer is formed from one or more of Platinum (Pt), Silver (Ag), and gold (Au).

17. (canceled)

18. A stretchable biopolymer-based heating pad, comprising:
a pollen-based substrate layer;
a cured polymeric layer or a metal layer on top of the pollen-based substrate layer, where, when present, the cured polymeric layer comprises a cured polymeric material and a metal; and
an encapsulation coating comprising a polymeric material that encapsulates the cured polymeric layer or the metal layer, wherein
the pollen-based substrate layer and the cured polymeric layer are patterned to provide a heating pad.

19. The stretchable biopolymer-based heating pad according to claim 18, wherein the encapsulation coating further encapsulates the pollen-based substrate layer.

20. The stretchable biopolymer-based heating pad according to claim 18, wherein, when present, the metal layer on top of the pollen-based substrate layer is formed from one or more of the group consisting of chromium (Cr), titanium (Ti), copper (Cu), platinum (Pt), silver (Ag), and gold (Au).

21. The stretchable biopolymer-based heating pad according to claim 20, wherein the metal layer on top of the pollen-based substrate layer is presented as a first layer and a second layer, with the first layer in direct contact with the pollen-based substrate layer.

22. (canceled)

23. (canceled)

24. The stretchable biopolymer-based heating pad according to claim 18, wherein, when present, the cured polymeric material is selected from one or more of a polyurethane and a silicone elastomer.

25. The stretchable biopolymer-based heating pad according to claim 18, wherein, when the cured polymeric material is present, the metal in the cured polymeric material is selected from one or more of the group consisting of platinum (Pt), silver (Ag), and gold (Au).

26. (canceled)

27. A method of forming a stretchable biopolymer-based heating pad as described in claim 18, wherein the method comprises the steps of:
(ci) providing a heating pad intermediate comprising:
a pollen-based substrate layer;
a cured polymeric layer or a metal layer on top of the pollen-based substrate layer, where, when present, the cured polymeric layer comprises a cured polymeric material and a metal; and
(cii) encapsulating at least the cured polymeric layer or the metal layer on top of the pollen-based substrate layer with an encapsulation coating comprising a polymeric material.

28. (canceled)

29. (canceled)

30. (canceled)
